(19) United States
(12) Patent Application Publication
(10) Pub. No.: US 2017/0196891 A1
(43) Pub. Date: Jul. 13, 2017

(54) FATTY ACID BILE ACID CONJUGATES FOR TREATMENT OF LIPODYSTROPHY

(71) Applicant: GALMED RESEARCH AND DEVELOPMENT LTD., Tel Aviv (IL)

(72) Inventors: Maya HALPERN, Kfar Tavor (IL); Allen BAHARAFF, Tel Aviv (IL)

(73) Assignee: GALMED RESEARCH AND DEVELOPMENT LTD., Tel Aviv (IL)

(21) Appl. No.: 15/313,640

(22) PCT Filed: May 31, 2015
(86) PCT No.: PCT/IL2015/050561

§ 371 Date: Nov. 23, 2016

Related U.S. Application Data
(60) Provisional application No. 62/006,178, filed on Jun. 1, 2014.

(51) Int. Cl. A61K 31/575; A61K 45/06

(52) U.S. Cl. CPC A61K 31/575; A61K 47/48038; A61K 45/06

(57) ABSTRACT

Provided are methods of treating and/or reducing risk of lipodystrophy using compositions including at least one fatty-acid/bile-acid conjugate (FABAC). Further provided are methods of treating HIV-associated lipodystrophy using said FABACs.

### FIELD OF THE INVENTION

The present disclosure relates generally to compositions comprising a fatty-acid/bile-acid conjugate (FABAC) for treatment of lipodystrophy or associated conditions.

### BACKGROUND OF THE INVENTION

Lipodystrophy is a common name for disorders characterized by selective loss of adipose tissue (body fat) from various body regions and/or accumulation of excess fat in other areas. Localized fat loss from one area, such as the face, is called lipoatrophy. The extent of fat loss can range from very small areas on one part of the body to near total absence of adipose tissue from the entire body. In addition, patients may have either severe metabolic complications or mere cosmetic problems. Lipodystrophy associated with severe fat loss may contribute to metabolic complications related to insulin resistance, such as diabetes mellitus, high levels of serum triglycerides and fatty liver (hepatic steatosis).

Lipodystrophy may be either congenital (such as familial partial lipodystrophy or Beradinelli-Seip syndrome) or acquired (e.g. associated with various types of illnesses or drugs).

Acquired lipodystrophies are caused by medications, autoimmune mechanisms or may be idiopathic. Acquired lipodystrophies include lipodystrophy in HIV-infected patients (LD-HIV) which may be induced by highly active antiretroviral therapy (HAART), acquired generalized lipodystrophy (AGL), acquired partial lipodystrophy (APL) and localized lipodystrophy. Acquired lipodystrophies do not have a direct genetic basis.

Acquired lipodystrophies have been prevalent for more than a century. Nevertheless, little progress has been made in understanding the underlying pathogenetic mechanisms and methods of treatment of acquired lipodystrophy. For example, despite more than 100,000 patients in the United States being affected by LD-HIV, the exact mechanism of HIV-associated lipodystrophy is not fully elucidated.

There is evidence indicating that HIV-associated lipodystrophy can be caused either by anti-retroviral therapy or by the HIV infection itself in the absence of anti-retroviral medication. In some cases, HIV-associated lipodystrophy appears after the patient received HIV-1 protease inhibitor-containing HAART for two years or more. Most patients of HIV-associated lipodystrophy gradually lose subcutaneous fat from the arms, legs, and face. Some areas of the body are spared, and some patients accumulate excess fat in other areas manifesting as “buffalo hump” (enlargement of the dorsocervical fat pad), double chin, and increased waist circumference. In some cases the fat loss worsens with ongoing HAART therapy and does not reverse on discontinuation of protease inhibitors. Between 30% and 70% of the patients develop non-alcoholic fatty liver disease (NAFLD), some progress to cirrhosis (Lionel Piroth, AIDS Reviews, 2005, 197-209). In addition, many patients develop hypertriglyceridemia and diabetes mellitus. In many patients, protease inhibitors and nucleoside reverse transcriptase inhibitors (NRTI) are implicated in causing lipodystrophy. Since protease inhibitors or nucleoside reverse transcriptase inhibitors are usually given together as part of the HAART, the individual effects of these drugs remains unclear.

Currently, the only available treatments for lipodystrophy are maintaining a low-carbohydrate, low-fat diet, and surgical treatments to replace lost fat or remove excess fat. Drugs from the glitazone family, such as rosiglitazone and pioglitazone, have been suggested for evaluation in the treatment of lipodystrophy. Myalept® (metreleptin) is indicated for ameliorating leptin deficiency in people with congenital or acquired generalized lipodystrophy. However, this drug is contraindicated in patients with HIV-related lipodystrophy or metabolic disease, and has been associated with marked side effects including an increased risk for lymphoma and loss of natural leptin activity due to development of neutralizing antibodies. There is therefore an unmet need for compositions and methods useful in treatment of lipodystrophy and/or associated conditions.

Fatty acid bile salt conjugates, referred to also as Fatty Acid Bile Acid Conjugates (FABACs), are a family of synthetic molecules that may be used to improve conditions related to bile acids or cholesterol metabolism. FABACs are believed to lower blood cholesterol concentration, reduce liver fat levels and dissolve gallstones (Gilat et al., Hepatology 2003; 38: 436-442; and Gilat et al., Hepatology 2002; 35: 597-600).

U.S. Pat. Nos. 6,384,024, 6,395,722, 6,589,946 disclose use of certain FABACs in dissolving cholesterol gallstones in bile and treating arteriosclerosis. These and additional FABACs were disclosed in U.S. Pat. Nos. 7,501,403, 8,975,246 and 8,110,564 for use in treating fatty liver, in reducing blood cholesterol levels and in treating hyperglycemia, diabetes, insulin resistance and obesity. Nowhere in the art is it disclosed or suggested that FABACs may be useful for prevention, treatment or attenuation of lipodystrophy.

### SUMMARY OF THE INVENTION

The present invention relates to treatment, amelioration or prevention of lipodystrophy and/or associated conditions using fatty acid bile acid conjugates (FABACs) or compositions comprising same. According to some embodiments, lipodystrophy is acquired lipodystrophy. According to some embodiments, lipodystrophy is Human Immunodeficiency Virus (HIV)-associated lipodystrophy.

According to some embodiments, HIV-associated lipodystrophy (LD-HIV) is induced by HIV infection. According to some embodiments, HIV-associated lipodystrophy is induced by treatment with anti-retroviral therapy. According to some embodiments, anti-retroviral therapy refers to anti-retroviral drugs. According to some embodiments, anti-retroviral drugs relevant to acquired HIV-associated lipodystrophy include: HIV-1 protease inhibitors (PIs), Nucleoside reverse transcriptase inhibitors (NRTIs), Non-nucleoside Reverse Transcriptase Inhibitors (NNRTIs), Fusion Inhibitors, Entry Inhibitors such as, but not limited to, CCRS co-receptor antagonist, HIV integrase strand transfer inhibitors and combinations thereof. According to some embodiments, anti-retroviral drugs relevant to acquired HIV-associated lipodystrophy include: reverse transcriptase inhibitors (RTIs), protease inhibitors and a combination thereof.

Without wishing to be bound by any theory or mechanism, administering the FABACs of the invention or a composition comprising same substantially simultaneously, concurrently, alternately, sequentially, successively or according to an overlapping schedule with at least one anti-retroviral drug prevents or ameliorates lipodystrophy and/or associated conditions. According to some embodiments, lipodystrophy associated with HIV is associated with conditions other than non-alcoholic fatty liver disease (NAFLD) and the metabolic syndrome associated with it.

According to some embodiments, the present invention provides a method for reducing, ameliorating or preventing lipodystrophy induced by anti-retroviral drugs and/or associated conditions, the method comprises administering the FABACs of the invention or a composition comprising same to a subject in need thereof. As used herein, the term “subject in need thereof” refers to a subject afflicted with lipodystrophy or a subject at risk of being afflicted with lipodystrophy. According to some embodiments, a subject infected with HIV and/or a subject treated with anti-retroviral therapy is a subject at risk of being afflicted with lipodystrophy. Each possibility represents a separate embodiment of the present invention.

According to one aspect, the present invention provides a method of treating lipodystrophy in a subject in need thereof, the method comprising administering to said subject a therapeutically effective amount of a fatty acid bile acid conjugate (FABAC) of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond. Each possibility represents a separate embodiment of the present invention.

According to some embodiments, the present invention provides a method of treating lipodystrophy in a subject in need thereof, the method comprises administering to said subject a composition comprising a therapeutically effective amount of the FABAC of the invention and a pharmaceutically acceptable excipient, diluent or carrier. Each possibility represents a separate embodiment of the present invention. According to some embodiments, the bonding member is selected from the group consisting of: NH, P, S, O and a direct C—C or C═C bond. Each possibility represents a separate embodiment of the present invention. According to some embodiments, the bonding member is NH.

According to some embodiments, each of said one or two fatty acid radicals is a radical of a fatty acid selected from the group consisting of: arachidylic acid, stearic acid, behenic acid, palmitic acid, arachidonic acid, eicosapentaenoic acid and oleic acid. Each possibility represents a separate embodiment of the present invention. According to some embodiments, said one or two fatty acid radicals are radicals of arachidylic acid. Each possibility represents a separate embodiment of the present invention.

According to some embodiments, W represents two fatty acid radicals, each independently comprises 6-22 carbon atoms; and wherein each of said fatty acid radicals is independently bound to a bonding member X selected from the group consisting of: a heteroatom, a direct C—C bond and a C═C bond. According to some embodiments, W represents a single fatty acid radical.

According to some embodiments, the bile acid is selected from the group consisting of: cholic acid, ursodeoxycholic acid, chenodeoxycholic acid, deoxycholic acid, lithocholic acid and derivatives thereof. Each possibility represents a separate embodiment of the present invention. According to some embodiments, the bile acid is cholic acid.

According to some embodiments, the FABAC is 3β-arachidylamido-7α, 12α-dihydroxy-5β-cholan-24-oic acid.

According to some embodiments, the lipodystrophy is induced by at least one of: HIV infection and anti-retroviral therapy. Each possibility represents a separate embodiment of the present invention. According to some embodiments, the lipodystrophy is induced by at least one anti-retroviral drug.

In another aspect, there is provided a method of reducing the risk of Human Immunodeficiency Virus (HIV)-associated lipodystrophy, the method comprising administering to a subject in need thereof a therapeutically effective amount of a fatty acid bile acid conjugate (FABAC) of Formula I as defined herein.

In one embodiment, said conjugate of Formula I is in the form of a pharmaceutical composition comprising at least one of a pharmaceutically acceptable excipient, diluent or carrier. In another embodiment said subject is HIV-positive (or infected with HIV). In another embodiment said subject is treated with at least one anti-retroviral drug. In another embodiment said at least one anti-retroviral drug is administered substantially simultaneously, concurrently, alternately, sequentially, successively or according to an overlapping schedule to said administration of said conjugate. In another embodiment said FAB AC is 3β-arachidylamido-7α, 12α-dihydroxy-5β-cholan-24-oic acid, or a pharmaceutically acceptable salt thereof.

According to some embodiments, the present invention provides a pharmaceutical composition for use in treating lipodystrophy or in reducing the risk of Human Immunodeficiency Virus (HIV)-associated lipodystrophy, the composition comprising a therapeutically effective amount of a fatty acid bile acid conjugate (FABAC) of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond.

According to some embodiments, the composition of the invention comprises at least one of the FABACs of the invention as an active ingredient. According to some embodiments, the composition of the invention comprises said conjugate as an active ingredient and further comprises at least one of a pharmaceutically acceptable excipient, diluent or carrier. Each possibility represents a separate embodiment of the present invention.

In another embodiment said bonding member is selected from the group consisting of: NH, P, S, O and a direct C—C or C═C bond. In another embodiment said bonding member is NH. In another embodiment each of said one or two fatty acid radicals is a radical of a fatty acid selected from the group consisting of: arachidylic acid, stearic acid, behenic acid, palmitic acid, arachidonic acid, eicosapentaenoic acid and oleic acid. In another embodiment said one or two fatty acid radicals are radicals of arachidylic acid. In another embodiment W represents two fatty acid radicals, each independently comprising 6-22 carbon atoms; and wherein each of said fatty acid radicals is independently bound to a bonding member X selected from the group consisting of: a heteroatom, a direct C—C bond and a C═C bond. In another embodiment W represents a single fatty acid radical. In another embodiment said bile acid is selected from the group consisting of: cholic acid, ursodeoxycholic acid, chenodeoxycholic acid, deoxycholic acid, lithocholic acid and derivatives thereof. In another embodiment said bile acid is cholic acid. In another embodiment said FABAC is 3β-arachidylamido-7α, 12α-dihydroxy-5β-cholan-24-oic acid or a salt thereof. In another embodiment said composition is adapted for administration simultaneously, concurrently, alternately, sequentially, successively or according to an overlapping schedule with at least one anti-retroviral drug.

In another aspect the invention provides a pharmaceutical composition comprising: a) a FABAC of Formula I as defined herein, b) at least one anti-retroviral drug, and optionally c) at least one of a pharmaceutically acceptable excipient, diluent or carrier.

According to certain embodiments, the at least one anti-retroviral drug is selected from the group consisting of: emtricitabine (FTC), lamivudine (3TC), zalcitabine (dideoxycytidine), zidovudine (AZT), didanosine, tenofovir disoproxil fumarate, stavudine, abacavir sulfate, rilpivirine, etravirine, delavirdine, efavirenz, nevirapine, amprenavir, tipranavir, indinavir, saquinavir, saquinavir mesylate, lopinavir, ritonavir, fosamprenavir calcium, darunavir, atazanavir sulfate, nelfinavir mesylate, enfuvirtide, raltegravir, dolutegravir and combinations thereof. In another embodiment said FABAC is 3β-arachidylamido-7α, 12α-dihydroxy-5β-cholan-24-oic acid or a salt thereof. In another embodiment the composition is used in treating lipodystrophy or in reducing the risk of Human Immunodeficiency Virus (HIV)-associated lipodystrophy.

In another aspect there is provided a kit comprising: a) a therapeutically effective amount of a FABAC of Formula I as defined herein, and b) instructions for administering the FAB AC simultaneously, concurrently, alternately, sequentially, successively or according to an overlapping schedule with at least one anti-retroviral drug.

In another embodiment, the kit further comprises the at least one anti-retroviral drug. In another embodiment the at least one anti-retroviral drug is selected from the group consisting of: emtricitabine (FTC), lamivudine (3TC), zalcitabine (dideoxycytidine), zidovudine (AZT), didanosine, tenofovir disoproxil fumarate, stavudine, abacavir sulfate, rilpivirine, etravirine, delavirdine, efavirenz, nevirapine, amprenavir, tipranavir, indinavir, saquinavir, saquinavir mesylate, lopinavir, ritonavir, fosamprenavir calcium, darunavir, atazanavir sulfate, nelfinavir mesylate, enfuvirtide, raltegravir, dolutegravir and combinations thereof. In another embodiment, said FABAC is 3β-arachidylamido-7α, 12α-dihydroxy-5β-cholan-24-oic acid or a salt thereof.

According to some embodiments, the present invention provides methods for treatment, amelioration or prevention of HIV-associated lipodystrophy and/or associated conditions by administering the FABACs of the invention or compositions comprising same to a subject in need thereof.

According to some embodiments, the present invention provides a method to ameliorate and/or prevent lipodystrophy induced by at least one antiretroviral drug, the method comprises administering the FABACs of the invention or compositions comprising same to a subject in need thereof.

Further embodiments, features, advantages and the full scope of applicability of the present invention will become apparent from the detailed description given hereinafter. However, it should be understood that the detailed description, while indicating preferred embodiments of the invention, are given by way of illustration only, since various changes and modifications within the spirit and scope of the invention will become apparent to those skilled in the art from this detailed description.

### DETAILED DESCRIPTION

The present invention relates to methods of using fatty acid bile acid conjugates (FABACs) or compositions comprising same for treatment, attenuation or prevention of lipodystrophy and/or associated conditions. According to some embodiments, the present invention provides a method for treatment, attenuation or prevention of Human Immunodeficiency Virus (HIV)-associated lipodystrophy and/or associated conditions, the method comprises administration of the FABACs of the invention to a subject in need thereof. Each possibility represents a separate embodiment of the present invention.

According to one aspect, the present disclosure provides a method of treating lipodystrophy in a subject in need thereof, the method comprising administering to said subject a therapeutically effective amount of a FABAC of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond. Each possibility represents a separate embodiment of the present invention.

In another aspect, the invention provides a method of reducing the risk of Human Immunodeficiency Virus (HIV)-associated lipodystrophy, the method comprising administering to a subject in need thereof a therapeutically effective amount of a fatty acid bile acid conjugate (FABAC) of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond. In another aspect, there is provided a pharmaceutical composition for use in treating lipodystrophy or in reducing the risk of Human Immunodeficiency Virus (HIV)-associated lipodystrophy, the composition comprising a therapeutically effective amount of a fatty acid bile acid conjugate (FABAC) of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond.

In another aspect, the invention provides a pharmaceutical composition comprising:
a) a FABAC of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond,
b) at least one anti-retroviral drug, and optionally
c) at least one of a pharmaceutically acceptable excipient, diluent or carrier.

In another aspect, there is provided a kit comprising:
a) a therapeutically effective amount of a FABAC of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond, and
b) instructions for administering the FABAC simultaneously, concurrently, alternately, sequentially, successively or according to an overlapping schedule with at least one anti-retroviral drug.

### Fatty Acid Bile Acid Conjugates

As used herein, the terms “FABAC”, “FABACs”, “BAFACs”, “the FABACs” and “the FABACs of the invention” are used interchangeably and refer to conjugates of the Formula W-X-G (Formula I), wherein G represents a bile acid or a bile salt radical thereof, W represents one or two fatty acid radical(s) having 6-22 carbon atoms, and X represents a bonding member between said bile acid and the fatty acid radical(s). According to some embodiments, bonding member X includes, but is not limited to, NH, P, S, O or a direct C═C or C—C bond. Each possibility represents a separate embodiment of the present invention. FABACs are known in the art, and are described, for example, in U.S. Pat. Nos. 6,384,024, 6,395,722, and 6,589,946, the contents of which are incorporated herein by reference. According to some embodiments, the one or two fatty acid radical(s) comprise 8-22 carbon atoms, possibly 14-22 carbon atoms, preferably 18-22 carbon atoms. Each possibility represents a separate embodiment of the present invention. According to some embodiments, the FABACs of the invention refer to FABACs used to treat, ameliorate, prevent or reduce the risk of lipodystrophy and/or associated conditions. Each possibility represents a separate embodiment of the present invention.

A non-limiting general structure of FABACs is set forth below. According to a non-limiting example, a bile acid is conjugated (e.g. using an amide bond, for example at position 3) with 1-2 fatty acids of any of a number of chain lengths. According to exemplary embodiment, the FABAC of the invention is 3β-arachidylamido-7α, 12α, dihydroxy-5β-cholan-24-oic acid (Arachidyl Amido Cholanoic Acid; an amide conjugate of cholic acid with arachidic acid; also known as “Aramchol” or “C20 FABAC”) or 3β-stearylamido-7α, 12α, dihydroxy-5β-cholan-24-oic acid (Stearyl Amido Cholanoic Acid; an amide conjugate of cholic acid with stearic acid; also known as “Steamchol” or “C18 FABAC”). Each possibility represents a separate embodiment of the present invention.

According to some embodiments, the FABAC of the invention has the Formula I:

W-X-G (I)

wherein G represents a bile acid or a bile salt radical thereof; W represents one or two saturated or unsaturated fatty acid radicals having 6-22 carbon atoms; and wherein X represents a bonding member or a direct C—C or a C═C bond. According to some embodiments, X represents a bonding member selected from the group consisting of: a heteroatom, a direct C—C bond and a C═C bond. Each possibility represents a separate embodiment of the present invention. According to some embodiments, bonding member X is selected from the group consisting of: NH, P, S, O and a direct C═C or C—C bond. Each possibility represents a separate embodiment of the present invention. According to some embodiments, G represents a bile acid radical.

According to some embodiments, the use of FABACs having the Formula II:

W-X-G (I)

wherein G represents a bile acid or a bile salt radical thereof; W represents a fatty acid radical having 6-22 carbon atoms; and wherein X represents a bonding member comprising a heteroatom or a direct C—C or C═C bond; and n is an integer 1 or 2, is contemplated. Each possibility represents a separate embodiment of the present invention. According to some embodiments, the heteroatom is selected from the group consisting of: NH, P, S and O. Each possibility represents a separate embodiment of the present invention. In general, the term “heteroatom” includes atoms of any element other than carbon or hydrogen, preferred examples of which include nitrogen, oxygen, sulfur, and phosphorus.

According to one embodiment n is 1. According to another embodiment n is 2, and at each occurrence W is independently a fatty acid radical having 6-22 carbon atoms and X is independently a bonding member comprising a heteroatom or a direct C—C or C═C bond. Each possibility represents a separate embodiment of the present invention. In another embodiment, the bonding member of the FABAC is selected from the group consisting of NH, P, S, O, or a direct C—C or C═C bond. Each possibility represents a separate embodiment of the present invention. According to some embodiments, the term “Direct bond” refers, to a C—C (single) bond. In another embodiment, the term “Direct bond” refers to a C═C (double) bond. In another embodiment, more than one direct bond is utilized in the FABAC of the invention. In another embodiment, the bond between the bile acid and the fatty acid radical(s) is in the beta configuration. In another embodiment, the bond between the bile acid and the fatty acid radical(s) is in the alpha configuration. In another embodiment, the bonding member is other than an ester bond.

According to some embodiments, the bile acid or bile acid radical of the FABAC is selected from the group consisting of: cholic acid, ursodeoxycholic acid, chenodeoxycholic acid, deoxycholic acid, lithocholic acid and derivatives thereof. Each type of bile acid or radical thereof represents a separate embodiment of the present invention. The term “radical” as used herein means a chemical moiety comprising one or more unpaired electrons. According to some embodiments, the bile acid or bile acid radical of the FABAC is cholic acid.

According to some embodiments, the FABAC of the invention comprises a single fatty acid radical. The conjugation of the bile acid with the fatty acid radical may take place at various positions of the bile acid. In certain embodiments, the conjugation of the bile acid with the fatty acid radical is performed in a position of the bile acid nucleus selected from the group consisting of: 3, 6, 7, 12 and 24. Each possibility represents a separate embodiment of the present invention. In one embodiment, said conjugation is performed in position 3 of the bile acid nucleus.

According to other embodiments, the FABAC of the invention comprises two fatty acid radicals. According to some embodiments, the conjugation of each fatty acid radical to the bile acid nucleus is at two positions of the bile acid nucleus selected from the group consisting of: 3, 7, 12 and 24. Each possibility represents a separate embodiment of the present invention. According to a particular embodiment, the conjugation of the two fatty acid radicals is at positions 3 and 7 of the bile acid nucleus.

According to some embodiments, the fatty acid is a short-chain fatty acid. According to some embodiments, the chain length of the short-chain fatty acid is 6-8 carbons. According to some embodiments, the fatty acid is a medium-chain fatty acid. According to some embodiments, the chain length of the medium-chain fatty acid is 8-14 carbons. According to some embodiments, the chain length of the fatty acid is 14-22 carbons. According to some embodiments, the chain length of the fatty acid is 16-22 carbons. According to certain embodiments, other fatty acid length known in the art may be utilized. Each type of fatty acid or fatty acid radical represents a separate embodiment of the present invention. According to some embodiments, pharmaceutical compositions according to the disclosed methods comprise more than one type of FABAC.

According to some embodiments, the fatty acid is saturated. According to some embodiments, the fatty acid is unsaturated. According to some embodiments, the fatty acid is mono-unsaturated. According to some embodiments, the fatty acid is poly-unsaturated. Each possibility represents a separate embodiment of the present invention.

According to some embodiments, the one or two fatty acids or fatty acid radicals of the FABACs of the invention are independently selected from the group consisting of: behenic acid, arachidylic acid, stearic acid, and palmitic acid. Each possibility represents a separate embodiment of the present invention. An exemplary embodiment of a FABAC according to the present invention is presented in Formula III herein below. According to some embodiments, in Formula III n=20 or n=18. Each possibility represents a separate embodiment of the present invention.

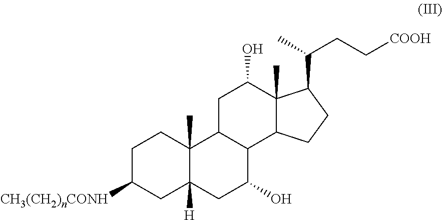

According to some embodiments, the one or two fatty acids or fatty acid radicals of the FABACs of the invention are unsaturated fatty acids or fatty acid radicals. Each possibility represents a separate embodiment of the present invention. According to some embodiments, the one or two unsaturated fatty acids or unsaturated fatty acid radicals of the FABAC are independently selected from the group consisting of: linolenic acid, eicosapentaenoic acid, docosahexaenoic acid, arachidonic acid, palmitoleic acid, oleic acid and elaidic acid. Each possibility represents a separate embodiment of the present invention.

As used herein, the term “conjugated fatty acid”, also known as “CFA”, refers to polyunsaturated fatty acids in which at least one pair of double bonds are separated by only one single bond.

According to some embodiments, the FABAC of methods and compositions of the present invention is selected from the group consisting of: 3β-behenylamido-7α, 12α-dihydroxy-5β-cholan-24-oic acid, 3β-arachidylamido-7α, 12α-dihydroxy-5β-cholan-24-oic acid, 3β-stearylamido-7α, 12α-dihydroxy-5β-cholan-24-oic acid and 3β-palmitylamido-7α, 12α-dihydroxy-5β-cholan-24-oic acid. Each possibility represents a separate embodiment of the present invention. According to exemplary embodiment, the FABAC of the invention is 3β-arachidylamido-7α, 12α, dihydroxy-5β-cholan-24-oic acid (Arachidyl Amido Cholanoic Acid; an amide conjugate of cholic acid with arachidic acid; also known as “Aramchol” or “C20 FABAC”). FABACs as described herein include pharmaceutically acceptable salts and/or derivatives thereof. Each possibility represents a separate embodiment of the present invention. As used herein, the term “bile acid derivative” includes bile acid salts with their pharmaceutically acceptable bases or acids as well as their diastereoisomeric and enantiomeric forms.

### Lipodystrophy and Related Methods

According to some embodiments, the disclosed compositions and methods provide amelioration and/or treatment and/or prevention and/or reduction of risk of lipodystrophy and/or associated conditions. Each possibility represents a separate embodiment of the present invention. As used herein, the term “lipodystrophy” relates to a disorder characterized by selective loss of adipose tissue (body fat) from various body regions and/or aberrant distribution of adipose tissue in various body regions. According to some embodiments, a subject afflicted with lipodystrophy may present associated conditions, such as, but not limited to, lipoatrophy. As used herein, the term “lipoatrophy” relates to a localized loss of adipose tissue from a specific body region, such as, but not limited to, the face. According to some embodiments, the associated conditions are other than NAFLD and/or the metabolic syndrome associated with NAFLD. According to some embodiments, the present disclosure provides a method of treatment and/or reduction of risk HIV-associated lipodystrophy by administration of the FABACs of the invention or compositions comprising same to a subject in need thereof. Each possibility represents a separate embodiment of the present invention.

As used herein, the term “treating” includes abrogating, substantially inhibiting, slowing or reversing the progression, substantially ameliorating clinical symptoms, or substantially preventing the appearance of symptoms and/or conditions associated with lipodystrophy. The treatment of lipodystrophy includes, according to some embodiments of the present invention, treatment of the symptoms and/or conditions described herein with respect to lipodystrophy. According to some embodiments, treatment of lipodystrophy comprises treatment of lipoatrophy.

Known indications suggested for FABC treatment include those disclosed in U.S. Pat. Nos. 6,384,024, 6,395,722, 6,589,946, 7,501,403, 8,110,564 and 8,975,246, as detailed herein. In contradistinction, the present specification surprisingly discloses that FABAC may provide therapeutic and/or prophylactic benefits in the management of lipodystrophy. It is herein disclosed for the first time that therapeutic regimens comprising FABACs are particularly advantageous in lipodystrophy patients. Accordingly, the methods of the invention advantageously comprise the step of identifying the subject as being afflicted with lipodystrophy, prior to administration of the FABACs of the invention.

The present invention for the first time provides for the treatment of new patient populations, not hitherto considered amenable for FABAC therapy. Thus, for example, the invention provides for the treatment of patients not manifesting the clinical symptoms associated with hitherto known pathologies or indications such as diabetes. In another non-limitative example, the methods of the invention advantageously include the treatment of subjects not manifesting liver inflammation. According to particular embodiments, the subject is not concurrently afflicted with bile gallstones or other pathologies or indications known to be treatable by FABACs. According to other advantageous embodiments, the methods of the invention comprise the step of identifying the subject as being at risk for developing lipodystrophy, prior to administration of the FABACs of the invention. By means of a non-limitative example, an HIV-infected (or HIV positive) subject may be determined amenable for treatment with the FABACs of the invention even in the absence of any other clinical symptoms or pathologies, to prevent, delay or ameliorate the development of HIV-induced lipodystrophy.

Lipodystrophy can generally be classified on the basis of the extent or pattern of fat loss (generalized or partial) as well as whether the disease is genetic or acquired. This classification scheme yields four major lipodystrophy subtypes: congenital generalized lipodystrophy (CGL), acquired generalized lipodystrophy (AGL), familial partial lipodystrophy (FPL), and acquired partial lipodystrophy (APL). HIV-associated lipodystrophy is commonly categorized as a type of APL. Exemplary lipodystrophies amenable for treatment by the methods of the invention include without limitation drug-induced (e.g. anti-retroviral drug-associated), infection-induced (e.g. HIV-associated), autoimmune and/or idiopathic lipodystrophies.

The diagnosis of lipodystrophy is mainly clinical. Distinction between various types of lipodystrophy can be made by medical history and physical examination. Skinfold thickness measurement, dual-energy X-ray absorptiometry (DXA, also known as DEXA), and whole body T-1 weighted magnetic resonance imaging (MRI) can provide information on the pattern of fat loss. For genetic lipodystrophies whose molecular basis is known, genetic testing, including prenatal diagnosis, is available for AGPAT2, BSCL2, LMNA, ZMPSTE24, and PPARG in clinical laboratories. Genotyping for other lipodystrophy genes such as CAV1, PTRF, AKT2, CIDEC, PLIN1 and PSMB8 is available on a research basis.

Clinical symptoms of lipodystrophy commonly include fat loss from the face and neck (e.g. CGL, AGL or APL), chest or trunk, upper extremities, lower extremities (e.g. APL) and/or intra-abdominal (e.g. CGL and AGL). Symptoms and signs may also include optional fat accumulation or sparing in the face and neck, hips and buttocks, lower extremities, or intra-abdominal. HIV-associated lipodystrophy may be manifested by one or more of the following symptoms: loss of subcutaneous fat in the arms, legs, face, and buttocks (lipoatrophy); fat gain in the abdomen and trunk, especially a gain in visceral fat (lipohypertrophy); dorsocervical fat pads, or “buffalo hump” on the back of the neck; and breast hypertrophy, usually in women.

According to some embodiments, the lipodystrophy is HIV-associated lipodystrophy. As used herein, the terms “HIV-associated lipodystrophy” and “LD-HIV” are used interchangeably and refer to lipodystrophy induced by Human Immunodeficiency Virus (HIV) infection and/or by anti-retroviral therapy. According to some embodiments, HIV-associated lipodystrophy is lipodystrophy induced by HIV infection and/or anti-retroviral drugs. Each possibility represents a separate embodiment of the present invention. According to some embodiments, the present invention provides a method of treating HIV-associated lipodystrophy in a subject in need thereof, the method comprising administering to said subject a therapeutically effective amount of a fatty acid bile acid conjugate (FABAC) of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond. Each possibility represents a separate embodiment of the present invention.

According to some embodiments, treatment of lipodystrophy relates to amelioration of at least part of lipodystrophy-associated conditions in a subject in need thereof. According to some embodiments, treatment of lipodystrophy comprises amelioration of lipoatrophy in a subject.

According to some embodiments, treatment of lipodystrophy relates to prevention and/or reduction of the risk of being afflicted with lipodystrophy and/or at least part of the lipodystrophy-associated conditions. Each possibility represents a separate embodiment of the present invention. According to some embodiments, the present disclosure provides a method for reducing risk of lipodystrophy and/or at least part of conditions associated with lipodystrophy, the method comprising administration of the FABACs of the invention or a composition comprising same. Each possibility represents a separate embodiment of the present invention.

According to some embodiments, the present disclosure provides a method for reducing risk of HIV-associated lipodystrophy and/or at least part of the conditions associated with or induced by said HIV-associated lipodystrophy, the method comprising administration of the FABACs of the invention or a composition comprising same. Each possibility represents a separate embodiment of the present invention. According to some embodiments, the present disclosure provides a method for reducing risk of HIV-associated lipodystrophy and/or at least part of the conditions induced by said HIV-associated lipodystrophy, the method comprising administration of the FABACs of the invention or a composition comprising same substantially simultaneously, concurrently, alternately, sequentially, successively or according to an overlapping schedule with at least one anti-retroviral drug. Each possibility represents a separate embodiment of the present invention.

According to some embodiments, anti-retroviral drugs relevant to acquired HIV-associated lipodystrophy include, without limitation: HIV-1 protease inhibitors (PIs), Nucleoside reverse transcriptase inhibitors (NRTIs), Non-nucleoside Reverse Transcriptase Inhibitors (NNRTIs), Fusion Inhibitors, Entry Inhibitors such as, but not limited to, CCR5 co-receptor antagonist, HIV integrase strand transfer inhibitors and combinations thereof. Each possibility represents a separate embodiment of the present invention.

Reverse-transcriptase inhibitors (RTIs) are a class of antiretroviral drugs used to treat HIV infection or AIDS that inhibit activity of reverse transcriptase, a viral DNA polymerase that is required for HIV replication. Nucleoside analog reverse-transcriptase inhibitors (NARTIs or NRTIs) compose the first class of antiretroviral drugs developed. In order to be incorporated into the viral DNA, NRTIs must be activated in the cell by the addition of three phosphate groups to their deoxyribose moiety, to form NRTI triphosphates. This phosphorylation step is carried out by cellular kinase enzymes. As used herein, this group further includes nucleotide analog reverse-transcriptase inhibitors (NtARTIs or NtRTIs), in which the conversion steps are skipped (e.g. tenofovir). NNRTIs, also known as “non-nucleosides” or “non-nukes” for short, attach themselves to reverse transcriptase and prevent the enzyme from converting RNA to DNA. In turn, HIV's genetic material cannot be incorporated into the healthy genetic material of the cell, and prevents the cell from producing new virus.

Non-limiting examples of NRTIs include: lamivudine and zidovudine, emtricitabine (FTC), lamivudine (3TC), abacavir and lamivudine, zalcitabine (dideoxycytidine), zidovudine (AZT), abacavir zidovudine and lamivudine, tenofovir disoproxil fumarate and emtricitabine, enteric coated didanosine, tenofovir disoproxil fumarate, stavudine, abacavir sulfate and a combination thereof.

Non-limiting examples of NNRTIs include: rilpivirine, etravirine, delavirdine, efavirenz, nevirapine and a combination thereof.

Protease inhibitors (PI) were designed to mimic the transition state of the protease's actual substrates. A peptide linkage consisting of —NH—CO— is replaced by a hydroxyethylen group (—CH2—CH(OH)—) which the protease is unable to cleave. HIV protease inhibitors fit the active site of the HIV aspartic protease and were rationally designed utilizing knowledge of the aspartyl protease's mode of action. The most promising transition state mimic was hydroxyethylamine which led to the discovery of the first protease inhibitor, saquinavir. Following that discovery, other HIV protease inhibitors were designed using the same principle.

Non-limiting examples of protease inhibitors include: amprenavir, tipranavir, indinavir, saquinavir, saquinavir mesylate, lopinavir and ritonavir, Fosamprenavir Calcium, ritonavir, darunavir, atazanavir sulfate, nelfinavir mesylate and a combination thereof.

Integrase inhibitors, also known as integrase strand transfer inhibitors (INSTIs), are a class of antiretroviral drug designed to block the action of integrase, a viral enzyme that inserts the viral genome into the DNA of the host cell. Since integration is a vital step in retroviral replication, blocking it can halt further spread of the virus.

Non-limiting examples of HIV integrase inhibitors include: raltegravir, dolutegravir and a combination thereof.

Entry inhibitors, also known as fusion inhibitors, are a class of antiretroviral drugs, typically used in combination therapy for the treatment of HIV infection. This class of drugs interferes with the binding, fusion and entry of an HIV virion to a human cell. By blocking this step in HIV's replication cycle, such agents slow the progression from HIV infection to AIDS.

Non-limiting example of a fusion inhibitor is enfuvirtide.

Without wishing to be bound by any theory or mechanism, administration of the FABACs of the invention to a subject afflicted with HIV substantially simultaneously, concurrently, alternately, sequentially, successively or according to an overlapping schedule with anti-retroviral drugs reduces the risk of lipodystrophy associated with HIV-infection or anti-retroviral drugs and/or associated conditions thereof in said subject.

The management of HIV/AIDS typically includes the use of multiple antiretroviral drugs in an attempt to control HIV infection. The use of multiple drugs that act on different viral targets (belonging to different classes of antiretroviral agents) is known as highly active antiretroviral therapy (HAART). Most current HAART regimens consist of three drugs: two NRTIs (“backbone”) and a PI, NNRTI or INSTI (“base”). Initial regimens use “first-line” drugs with a high efficacy and low side-effect profile. Exemplary initial regimens for adults and adolescents in the United States, are tenofovir/emtricitabine and raltegravir; tenofovir/emtricitabine and dolutegravir; abacavir/lamivudine (two NRTIs) and dolutegravir for patients who have been tested negative for the HLA-B*5701 gene allele tenofovir/emtricitabine, elvitegravir (an integrase inhibitor) and cobicistat (inhibiting metabolism of the former) in patients with good kidney function (gfr>70); and tenofovir/emtricitabine, ritonavir, and darunavir. According to certain embodiments, the FABAC of the invention are simultaneously, concurrently, alternately, sequentially, successively or according to an overlapping schedule with HAART treatment as described herein.

Anti retroviral drugs are commercially available and/or may be produced by well-known synthesis methods. Dosage and administration routes for HAART or other anti-retroviral therapy are well known and may be readily adjusted to the patient's condition by the treating physician.

In another aspect, there are provided kits or pharmaceutical packs comprising a FABAC of the invention, and instructions for administering the FABAC to a subject afflicted with, or, in other embodiments, at risk for developing, lipodystrophy. In another aspect, there is provided a kit comprising a therapeutically effective amount of a FABAC of the invention, and instructions for administering the FABAC simultaneously, concurrently, alternately, sequentially, successively or according to an overlapping schedule with at least one anti-retroviral drug. In a particular embodiment, the kit further comprises the at least one anti-retroviral drug.

According to some embodiments, the present invention provides a method of reducing and/or preventing lipoatrophy associated with lipodystrophy in a subject, the method comprises administering to said subject the FABACs of the invention or a composition comprising same. Each possibility represents a separate embodiment of the present invention. According to some embodiments, the present invention provides a method of reducing and/or preventing lipoatrophy in a subject afflicted with HIV-infection, the method comprises administering to said subject the FABACs of the invention or a composition comprising same substantially simultaneously, concurrently, alternately, sequentially, successively or according to an overlapping schedule with at least one anti-retroviral drug. Each possibility represents a separate embodiment of the present invention. According to some embodiments, lipoatrophy is facial lipoatrophy.

In another aspect, there is provided a method for determining whether a subject is amenable for treatment with a FABAC of the invention (e.g. a conjugate of Formula (I) or specifically Aramchol), comprising determining whether the subject is afflicted with lipodystrophy, wherein a subject identified as being afflicted with lipodystrophy is determined to be amenable for treatment with the FABAC. Determining whether the subject is afflicted with lipodystrophy is readily performed by well known diagnostic methods. Overall body fat, as well as fat content in specific organs, may be evaluated and compared to average values of healthy controls, wherein a significantly reduced fat level compared to control indicates fat loss in the test subject or organ and a significantly enhanced fat level compared to control indicates fat gain in the test subject or organ. Thus, for example, a significantly reduced total body fat content compared to healthy controls (e.g. significantly below the lower limit for healthy controls, as measured by DXA) indicates that said subject is afflicted with lipodystrophy.

DXA is an exemplary method for estimating body fat percentage, and determining body composition and bone mineral density. X-rays of two different energies are used to scan the body, one of which is absorbed more strongly by fat than the other. A computer can subtract one image from the other, and the difference indicates the amount of fat relative to other tissues at each point. A sum over the entire image enables calculation of the overall body composition. For example, DXA scans are typically performed before HIV treatment is started, and repeated annually to monitor for changes.

MRI scans provide a computer image of the tissues, muscle and bone in a cross-section of any part of the body. An MRI scan can show how fat is distributed—whether it is subcutaneous (under the skin) or visceral (around your central organs)—and is very accurate at measuring any changes. In the bioelectrical impedance analysis (BIA) method, two or more conductors are attached to a person's body and a small electric current is sent through the body. The resistance between the conductors will provide a measure of body fat between a pair of electrodes, since the resistance to electricity varies between adipose, muscular and skeletal tissue. Fat-free mass (muscle) is a good conductor as it contains a large amount of water (approximately 73%) and electrolytes, while fat is anhydrous and a poor conductor of electric current. Factors that affect the accuracy and precision of this method include instrumentation, subject factors, technician skill, and the prediction equation formulated to estimate the fat-free mass. It has mainly been used for HIV-related wasting but may also be useful in monitoring lipodystrophy.

Determining whether a subject is at risk of developing lipodystrophy is also within the scope of the skilled artisan, based on the presence of known risk factors as detailed herein (e.g. genetic markers or HIV infection). Determining the presence of HIV infection is routinely performed by various immunoassays (e.g. enzyme-linked immunosorbent assay, ELISA) or nucleic-acid based assays such as polymerase chain reaction (PCR).

According to some embodiments, the present disclosure provides a method for treating lipodystrophy and/or at least one associated condition thereof, the method comprising administering to a subject in need thereof a composition comprising a therapeutically effective amount of 3β-arachidylamido-7α, 12α-dihydroxy-5β-cholan-24-oic acid. Each possibility represents a separate embodiment of the present invention.

According to another aspect, the present disclosure provides a method of reducing the risk of HIV-associated lipodystrophy and/or associated conditions thereof, the method comprising administering to a subject a therapeutically effective amount of a fatty acid bile acid conjugate (FABAC) of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond. Each possibility represents a separate embodiment of the present invention.

According to some embodiments, the subject treated with the FABACs of the invention to reduce risk of HIV-associated lipodystrophy is a subject infected with Human Immunodeficiency Virus (HIV) or a subject afflicted with AIDS. Each possibility represents a separate embodiment of the present invention. According to some embodiments, the subject treated with the FABACs of the invention to reduce risk of HIV-associated lipodystrophy is a subject treated with at least one anti-retroviral drug. According to some embodiments, the at least one anti-retroviral drug is administered to the subject substantially simultaneously, concurrently, alternately, sequentially, successively or according to an overlapping schedule with said administration of said FABACs. Each possibility represents a separate embodiment of the present invention.

According to some embodiments, the FABACs of the invention are in the form of a pharmaceutical composition. According to some embodiments, the composition of the invention comprises one type of FABAC. According to some embodiments, the composition of the invention comprises Aramchol. According to other embodiments, the composition of the invention comprises a plurality of FABACs.

According to some embodiments, the FABACs of the invention are in the form of a composition (referred to as the composition of the invention) comprising a therapeutically effective amount of at least one of the FABACs of the invention. As used herein, the term “effective amount” means an amount of compound that is capable of inhibiting and/or reducing and/or attenuating and/or treating lipodystrophy and/or conditions associated therewith. According to some embodiments, lipodystrophy is HIV-associated lipodystrophy. The specific dose of a compound administered according to this invention will, of course, be determined by the particular circumstances surrounding the case including, for example, the compound administered, the route of administration, the physiological state of the subject, and the severity of the lipodystrophy or associated condition being treated. According to some embodiments, the composition is administered in several dosages over prolonged periods until a sufficient response has been achieved.

Non-limitative examples of suitable dosage and administration regimes are provided in the Examples section herein. For example, without limitation, an effective amount for oral administration to human subjects, e.g. HIV-infected subjects, may be a daily dose of 100-800 mg, typically 300-600 mg, e.g. a dose of 600 mg administered daily for a period of weeks, months or years.

### Pharmaceutical Compositions

Any suitable route may be used according to the methods of the present invention, to administer the composition of the invention to a subject.

According to some embodiments, suitable administration routes may be systemic routes. According to some embodiments, administering is administering systemically.

According to some embodiments, the composition is formulated for systemic administration.

According to another embodiment, administration systemically is through an enteral route. According to another embodiment, administration through an enteral route is oral administration. According to some embodiments, the composition is formulated for oral administration.

According to some embodiments, oral administration is in the form of hard or soft gelatin capsules, pills, capsules, tablets, including coated tablets, dragees, elixirs, suspensions, liquids, gels, slurries or syrups and controlled release forms thereof.

Suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

If desired, disintegrating agents, such as cross-linked polyvinyl pyrrolidone, agar, or alginic acid or a salt thereof, such as sodium alginate, may be added. Capsules and cartridges of, for example, gelatin for use in a dispenser may be formulated containing a powder mix of the compound and a suitable powder base, such as lactose or starch.

Solid dosage forms for oral administration include without limitation capsules, tablets, pills, powders, and granules. In such solid dosage forms, the active compound is admixed with at least one inert pharmaceutically acceptable carrier such as sucrose, lactose, or starch. Such dosage forms can also comprise, as it normal practice, additional substances other than inert diluents, e.g., lubricating, agents. In the case of capsules, tablets and pills, the dosage forms may also comprise buffering agents. Tablets and pills can additionally be prepared with enteric coatings. The term “enteric coating”, as used herein, refers to a coating which controls the location of composition absorption within the digestive system. Non-limiting examples for materials used for enteric coating are fatty acids, waxes, plant fibers or plastics.

Liquid dosage forms for oral administration may further contain adjuvants, such as wetting agents, emulsifying and suspending agents, and sweetening, flavoring and perfuming agents.

According to some embodiments, the present disclosure provides a pharmaceutical composition for use in treating lipodystrophy, the composition comprising a therapeutically effective amount of a fatty acid bile acid conjugate (FABAC) of Formula I:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond. Each possibility represents a separate embodiment of the present invention.

According to some embodiments, the present disclosure provides a pharmaceutical composition for use in treating lipodystrophy, the composition comprising a therapeutically effective amount of a 3β-arachidylamido-7α, 12α-dihydroxy-5β-cholan-24-oic acid.

The pharmaceutical composition may optionally further comprise additional active ingredients. According to particular embodiments, the pharmaceutical composition may further comprise one or more anti-retroviral drugs, e.g. PIs, NRTIs, NNRTIs, Fusion or Entry Inhibitors, HIV integrase strand transfer inhibitors and combinations thereof. In certain embodiments, the combination of anti-retroviral drugs is a HAART therapy, e.g. a combination of two NRTIs and a PI, NNRTI or INSTI. Each possibility represents a separate embodiment of the invention.

For, example, without limitation, the at least one anti-retroviral drug may be selected from the group consisting of: emtricitabine (FTC), lamivudine (3TC), zalcitabine (dideoxycytidine), zidovudine (AZT), didanosine, tenofovir disoproxil fumarate, stavudine, abacavir sulfate, rilpivirine, etravirine, delavirdine, efavirenz, nevirapine, amprenavir, tipranavir, indinavir, saquinavir, saquinavir mesylate, lopinavir, ritonavir, fosamprenavir calcium, darunavir, atazanavir sulfate, nelfinavir mesylate, enfuvirtide, raltegravir, dolutegravir and combinations thereof. Each possibility represents a separate embodiment of the invention.

According to other embodiments, the pharmaceutical composition comprises the FABAC of the invention (or in other embodiments a combination of FABACs of the invention) as sole active ingredient.

The terms “comprises”, “comprising”, “includes”, “including”, “having” and their conjugates, as used herein, mean “including but not limited to”. The terms “comprises” and “comprising” are limited in some embodiments to “consists” and “consisting”, respectively. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. In the description and claims of the application, each of the words “comprise” “include” and “have”, and forms thereof, are not necessarily limited to members in a list with which the words may be associated.

As used herein, the singular form “a”, “an” and “the” include plural references unless the context clearly dictates otherwise. For example, the term “a compound” or “at least one compound” may include a plurality of compounds, including mixtures thereof.

As used herein the term “about” refers to plus/minus 10% of the value stated. As used herein, the term “plurality” refers to at least two.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable combination or as suitable in any other described embodiment of the disclosure. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples. The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

A randomized, double-blinded, placebo-controlled trial examining the effects of Aramchol (3β-arachidylamido-7α, 12α, dihydroxy-5β-cholan-24-oic acid) in HIV-associated lipodystrophy patients is performed as follows.

50 HIV-positive subjects are randomized and orally administered 600 mg Aramchol or placebo. The treatment is administered daily and maintained for 16 weeks and up to 5 years. Total body fat is evaluated by DXA prior to treatment and after 16 weeks of treatment. Patients are monitored at regular intervals for side effects of Aramchol, symptoms of liver disease and serum biochemical and metabolic indices.

Toxicity is ranked as Grades I to 4, using common toxicity criteria (CTC) version 4.0 for scoring adverse events during therapy. Adverse events are defined as any adverse change from the patient's baseline (pre-treatment) condition. If any grade 2 adverse events (anemia, elevated serum CPK, hypoglycemia, decrease in serum bicarbonate, elevation in liver transaminases, elevation of bilirubin, anorexia, nausea, diarrhea) occur and persist on repeat testing for one week, Aramchol dosage is reduced from 600 to 300 and the patient is closely monitored. If the adverse events resolved and are not believed to be caused by Aramchol, the drug is restarted at a daily dose of 600 mg.

A sample size of 22 patients in each arm is chosen to provide a power 90% (or higher) with a β of 0.05. A two-tailed t-test is used to compare the differences between the two groups.

1.-38. (canceled)

39. A method of treating lipodystrophy in a subject in need thereof, the method comprising administering to said subject a therapeutically effective amount of a fatty acid bile acid conjugate (FABAC) of Formula I:
W-X-G (I)
wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond.

40. The method of claim 39, wherein said conjugate of Formula I is in the form of a pharmaceutical composition comprising at least one of a pharmaceutically acceptable excipient, diluent or carrier.

41. The method of claim 39, wherein said bonding member is selected from the group consisting of: NH, P, S, O and a direct C—C or C═C bond, each of said one or two fatty acid radicals is a radical of a fatty acid selected from the group consisting of: arachidylic acid, stearic acid, behenic acid, palmitic acid, arachidonic acid, eicosapentaenoic acid and oleic acid, and said bile acid is selected from the group consisting of: cholic acid, ursodeoxycholic acid, chenodeoxycholic acid, deoxycholic acid, lithocholic acid and derivatives thereof.

42. The method of claim 39, wherein said bonding member is NH, or wherein W represents two fatty acid radicals, each independently comprising 6-22 carbon atoms; and wherein each of said fatty acid radicals is independently bound to a bonding member X selected from the group consisting of: a heteroatom, a direct C—C bond and a C═C bond, or wherein W represents a single fatty acid radical.

43. The method of claim 39, wherein said one or two fatty acid radicals are radicals of arachidylic acid, or wherein said bile acid is cholic acid.

44. The method of claim 39, wherein said FABAC is 3β-arachidylamido-7α, 12α-dihydroxy-5β-cholan-24-oic acid.

45. The method of claim 39, wherein said lipodystrophy is induced by at least one of: HIV (Human Immunodeficiency Virus) infection and anti-retroviral therapy.

46. A method of reducing the risk of Human Immunodeficiency Virus (HIV)-associated lipodystrophy, the method comprising administering to a subject in need thereof a therapeutically effective amount of a fatty acid bile acid conjugate (FABAC) of Formula I:
W-X-G (I)
wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond.

47. The method of claim 46, wherein said conjugate of Formula I is in the form of a pharmaceutical composition comprising at least one of a pharmaceutically acceptable excipient, diluent or carrier.

48. The method of claim 46, wherein said subject is infected with HIV, or wherein said subject is treated with at least one anti-retroviral drug.

49. The method of claim 46, wherein said subject is treated with at least one anti-retroviral drug administered substantially simultaneously, concurrently, alternately, sequentially, successively or according to an overlapping schedule to said administration of said conjugate.

50. The method of claim 46, wherein said FABAC is 3β-arachidylamido-7α, 12α-dihydroxy-5β-cholan-24-oic acid, or a pharmaceutically acceptable salt thereof.

51. A pharmaceutical composition comprising:
a) a FABAC of Formula I:
W-X-G (I)
wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond,
b) at least one anti-retroviral drug, and optionally
c) at least one of a pharmaceutically acceptable excipient, diluent or carrier.

52. The pharmaceutical composition of claim 51, wherein the at least one anti-retroviral drug is selected from the group consisting of: emtricitabine (FTC), lamivudine (3TC), zalcitabine (dideoxycytidine), zidovudine (AZT), didanosine, tenofovir disoproxil fumarate, stavudine, abacavir sulfate, rilpivirine, etravirine, delavirdine, efavirenz, nevirapine, amprenavir, tipranavir, indinavir, saquinavir, saquinavir mesylate, lopinavir, ritonavir, fosamprenavir calcium, darunavir, atazanavir sulfate, nelfinavir mesylate, enfuvirtide, raltegravir, dolutegravir and combinations thereof.

53. The pharmaceutical composition of claim 51, wherein said FABAC is 3β-arachidylamido-7α, 12α-dihydroxy-5β-cholan-24-oic acid or a salt thereof.

54. A kit comprising:
a) a therapeutically effective amount of a FABAC of Formula I:
W-X-G (I)
wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond, and
b) instructions for administering the FABAC simultaneously, concurrently, alternately, sequentially, successively or according to an overlapping schedule with at least one anti-retroviral drug.

55. The kit of claim 54, further comprising the at least one anti-retroviral drug.

56. The kit of claim 55, wherein the at least one anti-retroviral drug is selected from the group consisting of: emtricitabine (FTC), lamivudine (3TC), zalcitabine (dideoxycytidine), zidovudine (AZT), didanosine, tenofovir disoproxil fumarate, stavudine, abacavir sulfate, rilpivirine, etravirine, delavirdine, efavirenz, nevirapine, amprenavir, tipranavir, indinavir, saquinavir, saquinavir mesylate, lopinavir, ritonavir, fosamprenavir calcium, darunavir, atazanavir sulfate, nelfinavir mesylate, enfuvirtide, raltegravir, dolutegravir and combinations thereof.

57. The kit of claim 54, wherein said FABAC is 3β-arachidylamido-7α, 12α-dihydroxy-5β-cholan-24-oic acid or a salt thereof.
